(19) United States
(12) Patent Application Publication
(10) Pub. No.: US 2023/0054008 A1
(43) Pub. Date: Feb. 23, 2023

(54) GRANULAR CONTEXT AWARE AUTOSUGGESTION OF DOCUMENT CODES

(71) Applicant: 3M INNOVATIVE PROPERTIES COMPANY, St. Paul, MN (US)

(72) Inventor: G. Edward Johnson, Bethesda, MD (US)

(21) Appl. No.: 17/796,898

(22) PCT Filed: Jan. 27, 2021
(86) PCT No.: PCT/IB2021/050638

§ 371 Date: Aug. 2, 2022

Related U.S. Application Data
(60) Provisional application No. 62/970,426, filed on Feb. 5, 2020.

(51) Int. Cl. G16H 40/20

(52) U.S. Cl. CPC G16H 40/20

(57) ABSTRACT

A method for evaluating an assignment of a code, the method including providing a code previously assigned to a document based on a plurality of sets of attributes, each attribute in the plurality of sets of attributes previously believed to affect the assignment of the code; eliminating the attributes not common to the sets of attributes in the plurality of sets of attributes; and evaluating the assignment of the code based on the remaining attributes.

### SUMMARY

In some aspects of the present description, a method for evaluating an assignment of a code is provided, the method including providing a code previously assigned to a document based on a plurality of sets of attributes, each attribute in the plurality of sets of attributes previously believed to affect the assignment of the code; eliminating the attributes not common to the sets of attributes in the plurality of sets of attributes; and evaluating the assignment of the code based on the remaining attributes.

In some aspects of the present description, a method for assigning a code to a document is provided, the method including providing previous assignments of the code, each previous assignment based on a presence of a set of attributes; grouping the attributes common to the previous assignments of the code in a new set of attributes; and assigning the code to the document based on a presence of the new set of attributes.

In some aspects of the present description, a computer-implemented method of assigning a code to a first document is provided, including providing a plurality of sets of attributes, each set of attributes in the plurality of sets of attributes corresponding to a previous assignment of the code to at least a second document; eliminating the attributes not common to the sets of attributes in the plurality of sets of attributes; creating a rule determining the assignment of the code to the first document based on the remaining attributes; and using the rule to determine if the code should be assigned to the first document.

### BRIEF DESCRIPTION OF THE DRAWINGS

FIG. 1A is a diagram of a system for generating rules guiding the automatic assignment of codes to a document, in accordance with an embodiment of the present description;

FIG. 1B is a flowchart illustrating a method for generating rules guiding the automatic assignment of codes to a document, in accordance with an embodiment of the present description;

FIG. 2 shows an example document with automatically assigned codes, in accordance with an embodiment of the present description;

FIG. 3A is a flowchart illustrating a method automatically assigning codes to a document, in accordance with an embodiment of the present description;

FIG. 3B is a flowchart illustrating a method automatically assigning codes to a document, in accordance with an alternate embodiment of the present description;

FIG. 3C is a flowchart illustrating a method creating rules for the automatic assignment of codes to a document, in accordance with an embodiment of the present description;

FIG. 4 is a table showing example rules regarding the automatic assignment of codes to a document, in accordance with an embodiment of the present description; and

FIG. 5 is an example rule regarding the automatic assignment of codes to a document, based on the data shown in FIG. 4, in accordance with an embodiment of the present description.

### DETAILED DESCRIPTION

In the following description, reference is made to the accompanying drawings that form a part hereof and in which various embodiments are shown by way of illustration. The drawings are not necessarily to scale. It is to be understood that other embodiments are contemplated and may be made without departing from the scope or spirit of the present description. The following detailed description, therefore, is not to be taken in a limiting sense.

There are automated tools in use for assigning codes to documents. For example, a natural language processing (NLP) software engine may be used to parse a medical document, looking for evidence and/or features within the document text that may be used as triggers for the automatic assignment of medical diagnostic and procedure codes to the medical document. When an automated tool or artificial intelligence (AI) engine is used to assign codes to a document, the results may be reviewed by a human operator, such as a coding expert, and the automatically-assigned codes may be corrected by the human or otherwise customized for a specific location or site. This feedback process may itself be automated or aided by software processes and tools.

For the purposes of this specification, the term “document” shall be defined broadly so as to include text documents, data repositories, text strings, or any other appropriate meaningful collection of data. For example, a “medical document” may in fact be a collection of data from various sources, including extracts from a doctor's transcribed medical notes, a patient database, data from an imaging laboratory, etc. In another example, a “document” may be a portion of a larger document, or a text string collected from a user interface of a remote station.

When an automatically assigned code is removed by an operator or software process, this may generate a “blacklist” entry or rule which may be used by the automated coding process to determine if the code should be assigned to documents in future instances. That is, the removal of an automatically assigned code may generate a rule that can be used by the coding process to suppress the assignment of the code in the future.

It should be noted that, although the term “blacklist” will be used in examples throughout this specification, the concepts described herein apply equally to the “whitelisting” of codes. While a blacklisting entry may prevent the assignment of a code to a document in certain circumstances and/or contexts, a whitelisting entry/rule is essentially the opposite. That is, a whitelist rule may assign a code that was not already assigned by the automated coding process, based on previous assignments of the code made in the review process. Both “blacklist” and “whitelist” rules may be thought of collectively as “assignment rules” (i.e., rules that are used to determine if a code should be assigned or removed from a document.) Any examples used herein discussing blacklist entries may be assumed to apply equally well to whitelist entries.

Preventing a code from being assigned to a document in a blacklist entry (or assigning the code in a whitelist entry) may not always be the best option. For example, just because a human coding expert working at a specific location removed a code from a document does not necessarily mean that the code should never be assigned to a document. The human expert may have had a very specific reason for removing or adding a code in a specific document that may not apply to all assignments of the code. For example, a coding expert reviewing code assignments in one diagnostic lab location (e.g., ABC Imaging Co.) may decide to suppress a certain medical code in a medical document for reasons very specific to ABC Imaging Co., which may not apply to other labs or locations. A better approach may be to examine a series of assignments of a specific code to documents, and to look at the granular (i.e., representing specific details of a document or the environment) context in which each code was blacklisted or whitelisted. In doing so, patterns of blacklisting/whitelisting may be established, and the “assignment rules” may be improved based on the patterns identified.

According to some aspects of the present description, a method for evaluating an assignment of a code (e.g., assigning a medical code to a medical document) includes providing a code previously assigned to a document based on a plurality of sets of attributes, each attribute in the plurality of sets of attributes previously believed to affect the assignment of the code; eliminating the attributes not common to the sets of attributes in the plurality of sets of attributes; and evaluating the assignment of the code based on the remaining attributes.

As used herein, the term “set(s) of attributes” may be assumed to mean a specific set of data items that represents the granular context of the document (and potentially other circumstances) in place at the time a code was assigned to or removed from a document. In the example of a diagnostic or procedure code being assigned to a medical document, this granular context may include, but not be limited to, the following items.

### Example Granular Context Element Examples for a Medical Code

CODE/CODE FAMILY: A code is the primary output of the coding process/coding engine. In the case of a medical document, it may be a diagnostic or procedure code. The Code Family is the grouping to which the code belongs. An example of a code family for a medical document is ICD10-CM (diagnosis codes) and a code in that code family is Z04.9. It is possible that a different code family also has a code named Z04.9, so Code Family may be important context.
PATH: Often, coding engines support multiple techniques for determining which codes should be assigned, and each technique may be given a “path” name. In some embodiments, a path name may be thought of as a location (e.g., a location in the cloud) where a database of information can be accessed. Some paths perform better than others for a particular code, or, sometimes support for a code is incomplete in a path and it may be desirable to remove the code from that path from the output without actually changing the path.
CODE ROLE: There are different sets of rules used for billing, depending on if the document is being coded for inpatient, outpatient, professional fee, or some other “code-role” In some embodiments, a coding engine may assign codes for different code roles, and it may be desirable to blacklist a code for a specific code role (but not for others).
REGION/SECTION: A medical document is divided into regions (sometimes called sections). These regions are of a known type, such as “Impression” or “History of Present Illness.” These regions often have titles that may be the same as the section type or may be a variant of it. It is possible that a path produces a code correctly when it suggests it from one region, but is often incorrect when it suggests it from another region. Being able to suppress a code when it comes from a specified region may help the accuracy of the engine.
EVIDENCE: The actual evidence found may, in some embodiments, be a key word or text string which triggers the assignment of a code. For example, the text string “Degenerative disc disease and spondylosis C5-6 and C6-7 levels” may trigger the assignment of a medical diagnostic code related to cervical disk degeneration.
SPECIALTY: In some embodiments, it may be helpful to take the medical specialty into account. For examples, a code may be appropriate for an Anesthesiology report but not be appropriate for a Dermatology report. In some embodiments, codes can be blacklisted/whitelisted by specialty.
DOCUMENT TYPE: The type of document being coded may affect the assignment of codes. For example, a “history and physical” (H&P) document may have different codes from a discharge summary.
PATIENT AGE/SEX: A patients age and/or sex may affect the assignment of codes. Some codes are often more appropriate for people of certain ages or sexes. That is, medical evidence found for a elderly man may, in most instances, mean something different than when the same evidence is found in a five-year-old child. In another simple example, a diagnostic code relating to pregnancy likely will not apply to a man.
DOCUMENT/REGION LENGTH: In some cases, the length of a document or a document region (e.g., short, average, long) may require special treatment.
IS ONLY CODE: In some embodiments, if there is only a single code on the document, it may not be desirable to remove the code.
OTHER DOCUMENT REGIONS: There is often a preferred region from which to determine codes to assign (such as “Impression”), but if there is no codable language in that region, other regions may be examined. Knowing which other regions exist in the document may help determine if a code should be blacklisted.
CUSTOMER, SITE, or PROVIDER: A particular customer, customer site, or provider (e.g., doctor) may not want a specific code to be assigned for reasons that apply only for them.
COMBINATION OF THE ABOVE: Any combination of the above elements (or others not shown) may be used to determine if a code should be assigned to a document. For example, a blacklist entry might look like “Suppress code A from Customer B when it comes from Document Type C and Region D, with Evidence E.”

This granular context may be captured by the coding process when a code is first assigned to a document. Stated another way, when the coding process determines that a certain code should be assigned, the granular context captured represents the state of the document and captures the evidence used for assigning the code at the time the assignment was made.

In some embodiments, the method described herein for evaluating an assignment of a code to a document examines a series of previously captured attribute sets for a given code, determines which attributes from each of the sets of attributes are common across all of the sets of attributes, and uses only those common attributes to determine if the code should be assigned to future documents. Stated another way, each time a code is assigned to a document, a granular context (similar to that described above) is captured for that assignment and stored. Sometime later, when the codes which were automatically assigned to the document are reviewed, changes may be made to the codes (e.g., a code may be deleted.) For example, in some embodiments, a user of the system may input information on the appropriateness of one or more of the assignments via a graphical user interface. In some embodiments, this input/feedback may be when a customer manually removes an autosuggested code (e.g., through a graphical user interface or similar tool). In other embodiments, this input/feedback may come from a nosologist (i.e., an expert in disease classification) or a coding analyst retrieving notes that a coding engine processed.

In some embodiments, the deletion of a code may create a blacklist “rule” or blacklist entry, which contains the identity of the code removed and the granular context when the code was originally assigned, including any “evidence” which was used as the basis for the original assignment. Any blacklist (or whitelist) rules thus created may be stored and used in future evaluations of the code to future documents. In some embodiments, the rules for evaluating the assignment of codes may be stored in a database.

However, these blacklist entries thus created may contain context elements that are very specific and unlikely to occur again, leading to unnecessary suppressions of the code. By examining several of the blacklist entries created for a specific code occurrence (i.e., examining the attributes captured in the granular context for each entry), and creating a new blacklist entry based only on the attributes found to be common to all of the sets of attributes, these unnecessary suppressions may be reduced or avoided. Examples of this are discussed in additional detail elsewhere herein.

In some embodiments, factors other than the remaining set of attributes may be used in the step of evaluating the assignment of the code. That is, the step of evaluating the assignment of the code to the document may be in part based on the remaining attributes (those found in common across the plurality of sets of attributes), and in part based on a new attribute not previously part of the sets of attributes. This new attribute may be based on an external reason (e.g., additional information not initially captured in the document), site practice/procedures, etc.

According to some aspects of the present description, a method for assigning a code to a document includes providing previous assignments of the code (e.g., a table or database of occurrences where a code was assigned to a document), each previous assignment based on a presence of a set of attributes (e.g., the granular context of the document at the time of the assignment); grouping the attributes common to the previous assignments of the code in a new set of attributes (i.e., creating a new entry for the code in question containing only those context elements which were found common to all of the occurrences); and assigning the code to the document based on a presence of the new set of attributes (i.e., when a document has the elements captured in the set of common attributes, we can make a decision about the assignment of the code).

It should be noted that phrases such as “assigning the code to the document based on a presence of the new set of attributes” may, in some embodiments, include suppression of the code for the particular document. That is, in the case of blacklist entries created based on common attributes, “assignment of the code” may actually include suppression of the code. Stated another way, the phrase “assign the code” may be interpreted to mean “make a decision on the assignment of the code”, which could include suppression of the code for blacklisting, and inclusion of the code for whitelisting.

In some embodiments, the method of assigning a code to a document may further include the step of creating a rule based on the new set of attributes, where the rule may be used to evaluate whether the code should be assigned to the document or not. In some embodiments, this rule may be stored in a database of such rules, to be used for future evaluations of the assignment of the code.

According to some aspects of the present description, a computer-implemented method of assigning a code to a first document includes providing a plurality of sets of attributes, each set of attributes in the plurality of sets of attributes corresponding to a previous assignment of the code to at least a second document; eliminating the attributes not common to the sets of attributes in the plurality of sets of attributes; creating a rule (e.g., a new blacklist or whitelist entry) determining the assignment (which may include suppression) of the code to the first document based on the remaining attributes; and using the rule to determine if the code should be assigned to the first document.

As stated elsewhere herein, each set of attributes used in the evaluation may include a context (e.g., a state of the document or automated system) at the time the code was previously assigned to a document. For example, a set of attributes may be captured at the moment the code is added to a blacklist entry (i.e., a rule or entry in a database indicating the code should not be assigned in at least a set of specific circumstances, such as may be captured in the set of attributes), or to a whitelist entry (i.e., a rule or entry in a database indicating the code should be assigned in at least a set of specific circumstances, when it had previously not been assigned). In some embodiments, the blacklist or whitelist entry may be created based on feedback received from a user.

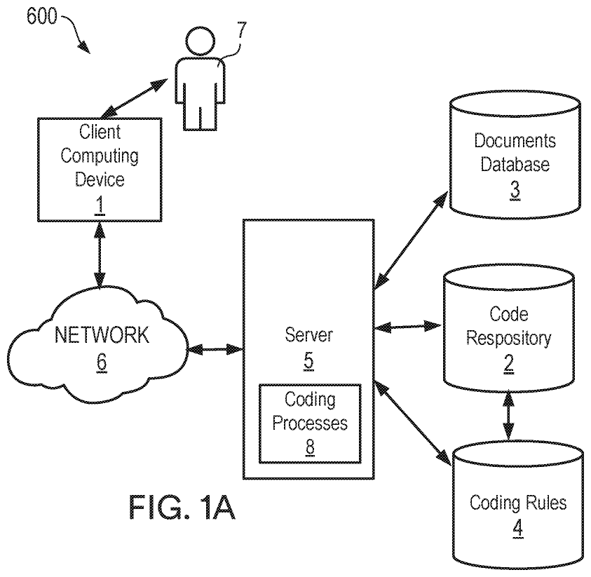

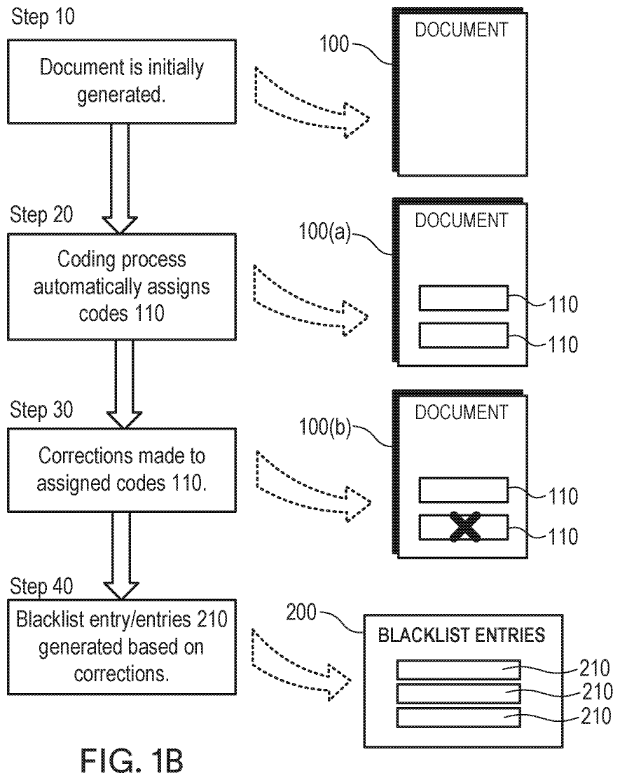

Turning now to the figures, FIG. 1A is a diagram of one possible embodiment of a system 600 for the automatic assignment of codes to a document, as described herein. In some embodiments, system 600 may include a client computing device 1 (e.g., a terminal, a portable computer, a handheld device, etc.) which accesses a computer server 5 over a network connection 6. The server 5 may provide access to various other systems and/or resources which may be used to perform a coding process (i.e., assign codes to documents). This coding process may be directed by a user 7, accessing server 5 via network 6, or may be partially or entirely automated, completed by one or more coding processes 8 running on server 5. In some embodiments, coding processes 8 may collect documents (or portions of documents) from a documents database 3 (e.g., a database of medical documents, such as transcribed physician notes, medical imaging data, patient history, etc.) Coding processes 8 may then access a database of coding rules 4. In some embodiments, coding rules 4 represent an embodiment of knowledge directing the assignment of codes to document or collection of documents. For example, coding rules 4 may be a knowledge engine, a machine learning model, or a set of data constructs which define “rules” for when certain codes should be assigned to certain documents. For example, coding rules 4 may contain a rule or guidance stating that when a term such as “Degenerative disc disease” is found in a medical document such as a doctor's transcribed notes, a certain medical diagnostic code (e.g., M50.321) related to disc degeneration should be assigned to the document by coding processes 8. Coding rules 4 may also contain “blacklisting” rules (which specify when a code should not be used, even if other rules say it should be) or “whitelisting” rules (which specify instances when a code should be used, even if other coding rules have not caused it to be assigned). In some embodiments, these “blacklisting” and “whitelisting” rules may be created (added to coding rules 4) when user 7 rejects a code that was automatically assigned by coding processes 8.

Consider the following medical coding example. User 7 accesses server 5 via a user interface on client computing device 1, executing coding processes 8. Coding processes 8 access document database 3, collecting documents or portions of documents related to a recent doctor's examination of a patient. Coding processes 8 analyze the documents (e.g., using a machine learning model, a natural language processing software module, or other appropriate automated process), looking for evidence to use in the diagnostic coding process. For example, coding processes 8 may parse the text of a document (which, as defined elsewhere herein, may be a collection of several documents, portions of a document, etc.) and find the phrases such as “family history of breast cancer”, “severe fibrocystic disease”, and “no evidence of malignancy” in a medical document (e.g., in the Diagnosis region of a document.) Based on this medical “evidence”, coding processes 8 may access the database of coding rules 4 and use the rules defined therein to determine the proper diagnostic code. The coding processes 8 use the evidence gathered from the medical document(s), the coding rules 4, and information from code repository 2 (which contains a listing of defined diagnostic codes) to determine one or more diagnostic codes to assign to the medical document. Given the evidence gathered in the above example, one such diagnostic code pulled from code repository 2 may be Z80.3, which relates to a “family history of malignant neoplasm of a breast.” In some embodiments, code repository 2 may represent an industry-defined listing of alphanumeric diagnostic codes used by physicians, insurance companies, and public health agencies, such as the International Classification of Diseases, Tenth Revision, Clinical Modification manual, also known as ICD-10-CM, although any appropriate code repository, manual, or database may be used.

After the coding processes 8 have used coding rules 4 and code repository 2 to determine one or more diagnostic codes to be assigned to the document, the information may be displayed to user 7 via client computing device 1. This display may occur at some time after the actual assignment of the code is completed, such as in a later code review process performed by user 7 (e.g., a trained coding expert). User 7 may decide they want to make a correction or amendment to the codes assigned automatically by coding processes 8. In the above example, user 7 may decide that code Z80.3 is not appropriate, and remove the code via a user interface on the client computing device. In some embodiments, that code removal may generate a new kind of coding rule called a “blacklist entry.” This blacklist entry may automatically be stored in the coding rules database 4 as a new rule, a rule which says not to use code Z80.3 under similar circumstances. In some embodiments, a set of attributes related to the document or the system environment may be captured at the time the blacklist entry is generated. That is, the blacklist entry that is automatically generated in such a review process will not only define which codes should not be used, but will also capture the specific conditions (a context of the document, for instance, including the evidence used) when the reviewer removed the code from the document.

Coding processes 8 may be any appropriate process used to assign codes from code repository 2 to documents, including fully automated processes and user-controlled processes using the system 600 as a tool to guide the user 7. Examples of fully automated processes may include, but are not limited to, natural language processing, machine learning models, and other computer-controlled processes. For example, a natural language processing (NLP) module may reside on server 5. Natural language processing is a subfield of linguistics and artificial intelligence concerned with the interactions between computer and human languages (such as the text in a medical document.) In some embodiments, natural language processing may be based on pre-defined rules based on grammars or heuristic rules for stemming (finding the root form of words being processed). In other embodiments, natural language processing may be based on a machine learning model. For example, a machine learning model may use statistical inference (e.g., inferring properties of a data set through analysis of samples or populations) to learn new “rules” through the analysis of a large body of text (e.g., a large collection of similar documents.)

FIG. 1B is a flowchart illustrating a method for generating rules (such as the blacklist entries defined previously) guiding the automatic assignment of codes to a document. In Step 10, a document 100 is initially generated. In the example of a medical scenario, a doctor may create document 100 by capturing notes from a medical examination of a patient. In Step 20, a coding process (e.g., an NLP coding engine or machine learning model) parses the document and automatically generates codes 110 to the document, producing coded document 100(a). For example, the coding process may analyze the document 100 and discover a phrase such as “Degenerative disc disease and spondylosis C5-6 and C6-7 levels” as evidence for the assignment of a code related to cervical disc degeneration, and assign the appropriate diagnostic code (e.g., M50.321/or and related codes) to create coded document 100(a). In Step 30, feedback is attained (e.g., during a review process such as that discussed in FIG. 1A) on the quality of the coded document 100(a) and corrections are made to the assigned codes 110 to produce corrected document 100(b). For example, a coding analyst at the site where document 100 was created may review the document (possibly using an automated tool designed for the task) and decide to remove one or more of codes 110 as being inappropriate. As described elsewhere, this correction/feedback may be done using an automated tool with a graphical user interface.

In Step 40, blacklist (and/or whitelist) entries 210 are created based on the corrections made in Step 30. In some embodiments, the blacklist entries 210 are stored in a database 200, which may contain other existing blacklist entries 210 or other rules (perhaps created by a separate process, such as the evaluation/analysis of document evidence) which can be used by the coding process to evaluate future codes to determined if they should be assigned. For example, in some embodiments, database 200 may be coding rules database 4 as described in FIG. 1A, and the contents of database 200 may include previously defined rules used in coding processes to assign codes to documents, along with blacklist entries 210 as created in Step 40.

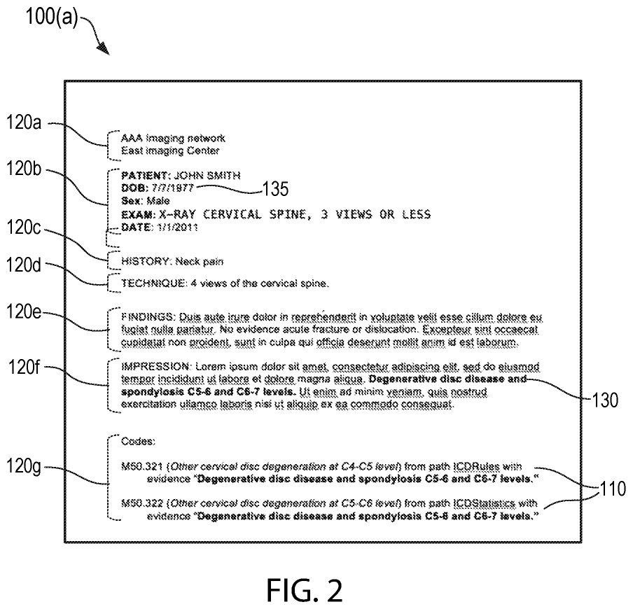

FIG. 2 shows an example medical document with assigned codes. For example, document shown in FIG. 2 may be document 100(a) created in Step 20 of FIG. 1B. Although the example shown in FIG. 2 represents a medical document, any appropriate type of document or dataset may be used without varying from the scope of the present document. In addition, document 100(a) as detailed in FIG. 2 is for illustration purposes, and is not intended to be limiting in any way. In the example embodiment of FIG. 2, document 100(a) may be divided into several document regions (i.e., sections) 120 (collectively, with letters designating specific types of regions, as detailed below). Example document regions 120 may include, but are not limited to:
120a: One or more regions may contain customer, site, or provider information, including a specific location (e.g., East Imaging Center) and/or network information (e.g., AAA Imaging Network).
120b: One or more regions may contain patient information, including patient name, age, and sex, as wells as information on the type of examination, and the date of the examination.
120c: One or more regions may contain medical history information, which may include current symptoms and/or pertinent prior medical history.
120d: One or more regions may contain information on the techniques and/or procedures used during the examination.
120e: One or more regions may contain the medical professional's findings and/or observations as a result of the examination.
120f: One or more regions may contain the medical professional's impression resulting from the examination. In the example of medical documents, this region is often an important source of “evidence” 130 that may be used during the coding process. In the example of FIG. 2, evidence 130 includes the phrase “Degenerative disc disease and spondylosis C5-6 and C6-7 levels,” which may result in diagnostic codes related to “cervical disc degeneration” to be assigned by the coding engine.
120g: One or more regions may contain codes 110 which may, in some embodiments, have been assigned automatically by an automated coding process (e.g., an NLP coding engine) in a subsequent process step (after the initial document 100 has been created). The codes 110 may include information on the path from which the codes were taken (e.g., ICDRules) and may include a copy of the specific evidence used in their assignment.

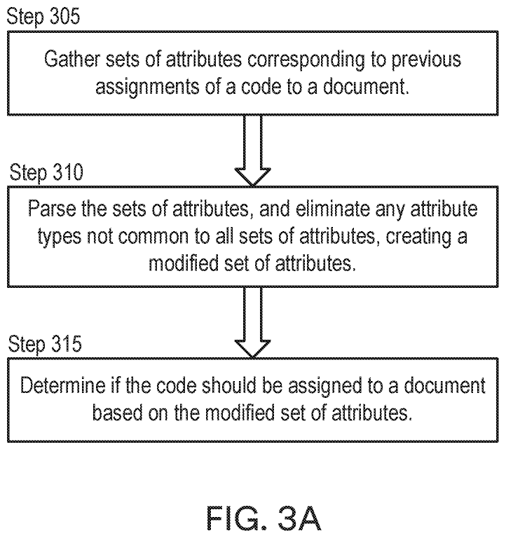

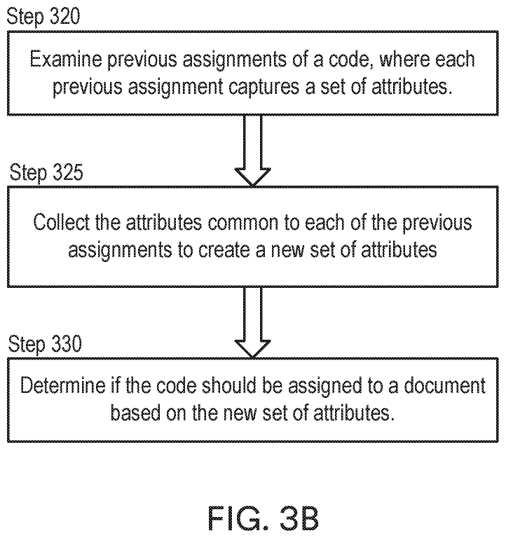

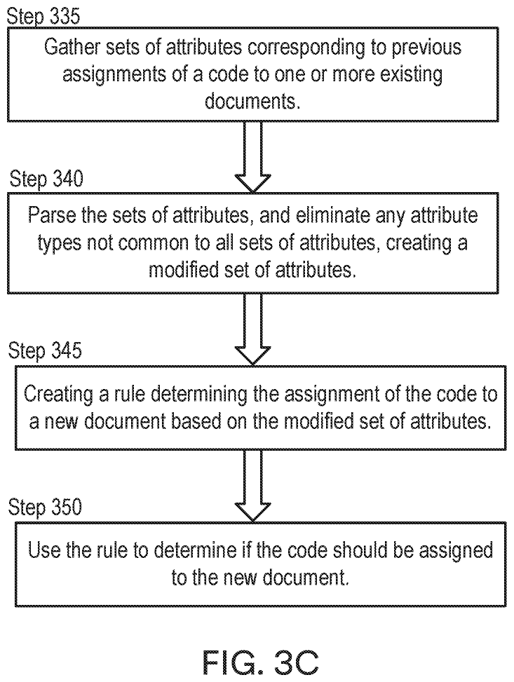

FIGS. 3A-3B are flowcharts illustrating alternate embodiments of a method for automatically assigning codes to a document, according to the present description. Turning first to FIG. 3A, in Step 305, sets of attributes representing the granular context captured during previous assignments of the code to a document are gathered. It is important to not that the term “previous assignments” may be interpreted such that it includes a codes removal from a document. That is, in some embodiments, the sets of attributes may represent either blacklist entries (corresponding to the previous removal of a code from a document) or whitelist entries (corresponding to the previous addition of a code to a document). In Step 310, all of the sets of attributes (all of the blacklist or whitelist entries) are parsed, and any attributes where are not common to all of the sets of attributes (not common to every entry) are eliminated, resulting in a modified set of attributes containing only those attributes which were found to be common across all sets of attributes. Finally, in Step 315, the modified set of attributes are used to determine if the code should be assigned to the document. That is, if the document being examined currently has a granular context which matches the attributes in the modified set of attributes, the code will be assigned to (for whitelisting) or removed from (for blacklisting) the document.

FIG. 3B is an alternate flowchart of a method similar to that of FIG. 3A, using different language for clarity. In Step 320, the method examines previous assignments of a code, where each previous assignment includes a set of attributes which captures the granular context of the document to which each of the previous code assignments was made. Again, for the purposes of this document, the phrase “assignment” (as in “assignment of a code”) shall be interpreted to include both the addition of a code to a document (e.g., a whitelisting addition of a code during a document review process) or the removal of a code from a document (e.g., a blacklisting removal of a code during a document review process.) In Step 325, the previous assignments are parsed and only the attributes which are common across all sets of attributes (i.e., across all of the previous assignments) are collected into a new set of attributes. Finally, in Step 330, the method determines if the code should be assigned to (added to or removed from) the document based on the new set of attributes.

FIG. 3C is another alternate flowchart of a method for the automatic assignment of codes to a document, including a step of creating a rule to be used for future assignments of the code. In Step 335, sets of attributes corresponding to previous assignments of the code are gathered. In Step 340, the various sets of attributes are parsed to create a modified set of attributes containing only those attributes which are common across all of the sets of attributes. In Step 345, a rule is created (where a rule may be a new blacklist entry or whitelist entry) based on the modified set of attributes which may be used to determine the assignment of the code to new documents. In Step 350, the new rule is used to determine if the code should be assigned to the new document.

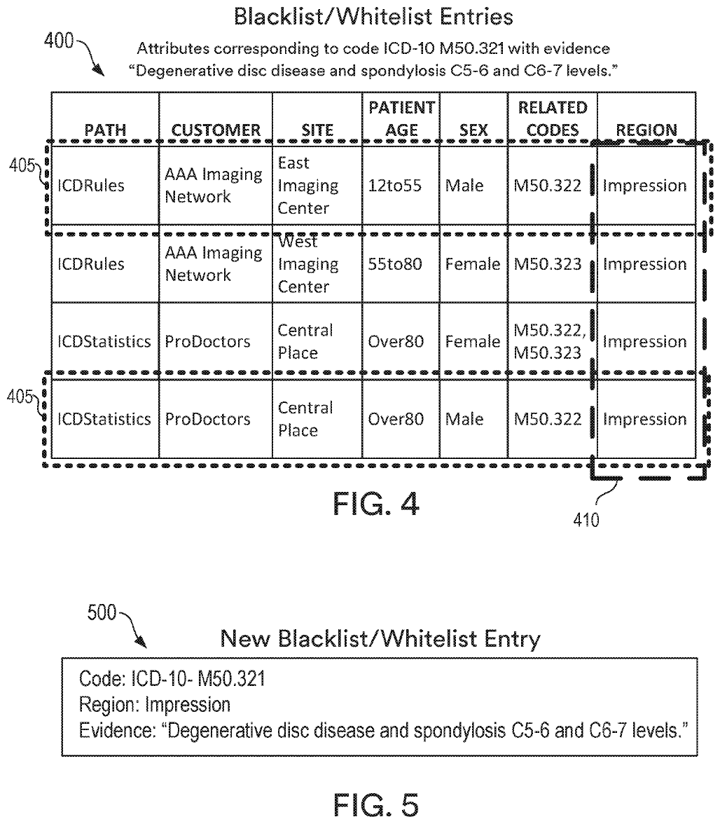

The previous description corresponding to FIGS. 3A-3C may be better understood with a specific example. FIGS. 4 and 5 provide such an example as used in the context of a medical document. As stated elsewhere herein, the use of a medical document example is not intended to be limiting in any way.

FIG. 4 is a table showing example blacklist entries (i.e., blacklist rules) related to the automatic assignment of a medical code to a medical document. In some embodiments, table 400 may be the database 200 of FIG. 1B, with each entry in table 400 corresponding to a blacklist rule 210 of FIG. 1B. The entries shown in the rows of FIG. 4 are examples only, for discussion purposes, and may not represent complete attribute sets as may be used in implementation. Each row 405 in table 400 represents a set of attributes (i.e., a specific set of data items representing a granular context of a document at the time the entry was created). As described at the top of FIG. 4, the example attribute sets shown in table 400 each relate to medical code ICD-10 M50.321, and each set of attributes has common evidence (e.g., text from the Impression region of one or more medical documents) “Degenerative disc disease and spondylosis C5-6 and C6-7 level.” Each row 405 of table 400 corresponds to a “set of attributes” as used in FIGS. 3A-3C.

As discussed in FIGS. 3A-3C, each row 405 (i.e., each “set of attributes”) is parsed to determine those attributes that are common to all rows 405. In the example of FIG. 4, the only attribute common across all rows 405 of table 400 is the “REGION” attribute, which is equal to “Impression” in each case. More simply stated, when looking at the entire table and each set of attributes 405, the “REGION” attribute was set to “Impression” each time the code M50.321 was blacklisted (removed during the document review process). Based on this set of common attributes, a new blacklist entry 500 may be created, as shown in FIG. 5.

In some embodiments, new blacklist entry 500 may replace all of the previous blacklist entries (rows 405 in FIG. 4). New entry 500 can now be applied to evaluate future assignments of code M50.321 (from path ICD-10) to a medical document. If the automated coding process has assigned code M50.321 based on the evidence “Degenerative disc disease and spondylosis C5-6 and C6-7” discovered in the “Impression” region of the document, this new blacklist entry 500 will cause the code to be removed from the document (for a blacklisting situation).

Terms such as “about” will be understood in the context in which they are used and described in the present description by one of ordinary skill in the art. If the use of “about” as applied to quantities expressing feature sizes, amounts, and physical properties is not otherwise clear to one of ordinary skill in the art in the context in which it is used and described in the present description, “about” will be understood to mean within 10 percent of the specified value. A quantity given as about a specified value can be precisely the specified value. For example, if it is not otherwise clear to one of ordinary skill in the art in the context in which it is used and described in the present description, a quantity having a value of about 1, means that the quantity has a value between 0.9 and 1.1, and that the value could be 1.

Terms such as “substantially” will be understood in the context in which they are used and described in the present description by one of ordinary skill in the art. If the use of “substantially equal” is not otherwise clear to one of ordinary skill in the art in the context in which it is used and described in the present description, “substantially equal” will mean about equal where about is as described above. If the use of “substantially parallel” is not otherwise clear to one of ordinary skill in the art in the context in which it is used and described in the present description, “substantially parallel” will mean within 30 degrees of parallel. Directions or surfaces described as substantially parallel to one another may, in some embodiments, be within 20 degrees, or within 10 degrees of parallel, or may be parallel or nominally parallel. If the use of “substantially aligned” is not otherwise clear to one of ordinary skill in the art in the context in which it is used and described in the present description, “substantially aligned” will mean aligned to within 20% of a width of the objects being aligned. Objects described as substantially aligned may, in some embodiments, be aligned to within 10% or to within 5% of a width of the objects being aligned.

All references, patents, and patent applications referenced in the foregoing are hereby incorporated herein by reference in their entirety in a consistent manner. In the event of inconsistencies or contradictions between portions of the incorporated references and this application, the information in the preceding description shall control.

Descriptions for elements in figures should be understood to apply equally to corresponding elements in other figures, unless indicated otherwise. Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A method for evaluating an assignment of a code, the method comprising:
providing a code previously assigned to a document based on a plurality of sets of attributes, each attribute in the plurality of sets of attributes previously believed to affect the assignment of the code;
eliminating attributes not common to the sets of attributes in the plurality of sets of attributes; and
evaluating the assignment of the code based on the remaining attributes.

2. The method for evaluating an assignment of a code of claim 1, wherein the step of evaluating the assignment of the code is further based on a new attribute.

3. The method for evaluating an assignment of a code of claim 1, further comprising creating a rule based on the remaining attributes, the rule to be used in a future evaluation of a second assignment of the code to a second document.

4. The method for evaluating an assignment of a code of claim 3, further comprising saving the rule to a database.

5. The method for evaluating an assignment of a code of claim 1, wherein each set of attributes in the plurality of sets of attributes comprises a granular context for the document at a time corresponding to an instance when the code was previously assigned to the document.

6. The method for evaluating an assignment of a code of claim 1, further comprising receiving, via a graphical user interface, information from a user on an appropriateness of a previous assignment of the code.

7. The method for evaluating an assignment of a code of claim 1, wherein each set of attributes in the plurality of sets of attributes is created when the code is added to a blacklist or a whitelist entry.

8. A method for assigning a code to a document, the method comprising:
providing previous assignments of the code, each previous assignment based on a presence of a set of attributes;
grouping attributes common to the previous assignments of the code in a new set of attributes; and
assigning the code to the document based on a presence of the new set of attributes.

9. The method for assigning a code to a document of claim 8, wherein the set of attributes corresponding to each previous assignment comprises a document context corresponding to the previous assignment.

10. The method for assigning a code to a document of claim 8, wherein the previous assignments of the code may include blacklisting the code or whitelisting the code.

11. The method for assigning a code to a document of claim 8, wherein the step of assigning the code to the document comprises creating a rule based on the new set of attributes, and using the rule to evaluate whether the code should be assigned to the document.

12. The method for assigning a code to a document of claim 11, wherein the rule is stored in a database for future use.

13. A computer-implemented method of assigning a code to a first document, comprising:
providing a plurality of sets of attributes, each set of attributes in the plurality of sets of attributes corresponding to a previous assignment of the code to at least a second document;
eliminating attributes not common to the sets of attributes in the plurality of sets of attributes;
creating a rule determining an assignment of the code to the first document based on the remaining attributes; and
using the rule to determine if the code should be assigned to the first document.

14. The computer-implemented method of assigning a code to a first document of claim 13, wherein each set of attributes in the plurality of sets of attributes comprises a context for the at least a second document at a time corresponding to an instance when the code was previously assigned to the at least a second document.

15. The computer-implemented method of assigning a code to a first document of claim 13, wherein each set of attributes in the plurality of sets of attributes is created when the code is added to a blacklist entry.

16. The computer-implemented method of assigning a code to a first document of claim 15, wherein the code is added to the blacklist entry based on feedback received from a user.

17. The computer-implemented method of assigning a code to a first document of claim 13, wherein each set of attributes in the plurality of sets of attributes is created when the code is added to a whitelist entry.

18. The computer-implemented method of assigning a code to a first document of claim 17, wherein the code is added to a whitelist entry based on feedback received from a user.
